(19) United States
(12) Patent Application Publication
(10) Pub. No.: US 2004/0042975 A1
(43) Pub. Date: Mar. 4, 2004

(54) Hair spray composition

(75) Inventors: David C. Streuli, Wayne, NJ (US); Linda C. Foltis, Nutley, NJ (US)

Correspondence Address: INTERNATIONAL SPECIALTY PRODUCTS, Attn: William J. Davis, Esq., Legal Dept., BLDG.10, 1361 Alps Road, Wayne, NJ, 07470, US

(73) Assignee: ISP INVESTMENTS INC.

(21) Appl. No.: 10/643,238

(22) Filed: Aug. 18, 2003

Related U.S. Application Data
(60) Provisional application No. 60/406,836, filed on Aug. 29, 2002.

(51) Int. Cl. A61K 7/06; A61K 7/11; A61K 9/00

(52) U.S. Cl. 424/47; 424/70.11

(57) ABSTRACT

A hair spray composition includes an isobutylene/ethylmaleimide/hydroxyethylmaleimide copolymer in a propellant mixture of (a) a hydrofluorocarbon, and (b) propane/isobutane which provides high pressure in a 55% VOC system.

### CROSS REFERENCE TO RELATED APPLICATIONS

This application corresponds to U.S. Provisional Patent Application Serial No. 60/406,836, filed on Aug. 29, 2002, the complete disclosure of which is hereby incorporated by reference.

### BACKGROUND OF THE INVENTION

This invention relates to hair spray compositions, and, more particularly, to a 55% VOC hair spray composition including a defined copolymer and propellant mixture.

### SUMMARY OF THE INVENTION

What is described herein is a hair spray composition comprising an isobutylene/ethylmaleimide/hydroxyethylmaleimide copolymer as film-former, water and alcohol, and a propellant admixture of (a) a hydrofluorocarbon and (b) propane/isobutane.

In the preferred embodiment of the invention, the 55% VOC hair spray composition includes, by wt., about 4% of the copolymer, about 6-12% water, about 45% ethanol, and 31% of (a) and 6% of (b) (50/50 wt. admixture) which provides high pressure and excellent hair styling.

### DETAILED DESCRIPTION OF THE INVENTION

Ulmer, H., in U.S. Pat. No. 6,025,501, described the preparation of derivatized polymers of α-olefin-maleic anhydride-alkyl half-ester or full acid. One such compound is isobutylene/ethylmaleimide/hydroxyethylmaleimide copolymer, sold in a 60% aqueous solution as Aquaflex® FX-64 (International Specialty Products Inc.). These copolymers are used in the hair spray compositions herein in combination with a defined propellant system.

The propellant in the 55% VOC hair styling spray of the invention is an admixture of a hydrofluorocarbon, e.g. 1,1-difluoroethane, Dymel® 152a (Dupont) and a 50/50 by wt. mixture of propane/isobutane or butane (A-70), which provides high pressure e.g. 70 psi, in a 55% VOC system.

This invention includes a polymeric concentrate, high pressure propellant blend in combination with a Summit SV-92/97 with V-94 actuator to form an audible high velocity spray. Although the spray is forceful, the mean particle size is around 60 μm. This system allows for the spray to be used as a styling tool to create styles. This invented application has designed the spray to reach the root of the hair allowing for a more dispersed spray pattern on the hair, resulting in a drier spray despite the fact that the formulation contains up to 10% water. This spray application to the root of the hair provides root lifting and apparent volume on the hair. Formulation has a very low tack phase and can therefore be used to aid in hair cutting. This styling spray may be used on wet or dry hair.

A preferred composition is shown in Table 1 below.

TABLE 1

| Ingredients | |
|---|---|
| Concentrate | % W/W |
| SD Alcohol 40-B (200 proof) | 45.50 |
| Deionized Water | 6.40 |
| Isobutylene/Ethylmaleimide/ | 10.00 |
| Hydroxyethylmaleimide Copolymer | |
| (4% active) | |
| Phenyl Trimethicone | 0.10 |
| Cyclopentasiloxane | 0.15 |
| Ammonium Hydroxide | 0.25 |
| Monoethanolamine (MEA) Borate (and) | 0.25 |
| Monoisopropanolamine (MIPA) Borate | |
| (Monacor BE) | |
| Fragrance | 0.10 |
| Propellant | |
| Hydrofluorocarbon 152a (Dymel 152a) | 31.25 |
| Propane/Isobutane (A-70) (50/50 wt/wt) | 6.00 |
| | 100.00% |
| Resin Solids | 4.00% |

Procedure

1. Add alcohol main tank. Start mixing. Pre-dissolve MEA Borate/MIPA Borate and water; add to alcohol.

2. Add isobutylene/ethylmaleimide/hydroxyethylmaleimide copolymer and mix until completely dissolved.

3. Add phenyl trimethicone and cyclopentasiloxane separately; mixing until uniform after each addition.

4. Add the fragrance and ammonium hydroxide separately; mix until homogeneous.

5. Fill into cans and charge with Hydrofluorocarbon 152a, followed by the A-70.

Packaging

Valve Summit SV-92/97 (0.016”×0.020 Body x 0.013” VT 0.125” ID)

Actuator V-94, 0.025″ MB

Can Type Unlined Tinplate or Polyamide Imide lined Aluminum (reduce Ammonium Hydroxide and MEA Borate/MIPA Borate to 0.1% each if the lined aluminum can is used). Can pressure rating 2P/2Q (TBD).

Another preferred composition is shown in Table 2 below.

TABLE 2

| Ingredients | |
|---|---|
| Concentrate | % W/W |
| SD Alcohol 40-B (200 proof) | 45.50 |
| Deionized Water | 6.45 |
| Isobutylene/Ethylmaleimide/ | 10.00 |
| Hydroxyethylmaleimide Copolymer | |
| Cyclopentasiloxane | 0.20 |
| Ammonium Hydroxide | 0.25 |
| MEA Borate (and) MIPA Borate | 0.25 |
| Fragrance | 0.10 |
| Propellant | |
| Hydrofluorocarbon 152a | 31.25 |
| Propane/Isobutane (A-70) | 6.00 |
| | 100.00% |
| Resin Solids | 4.00% |

Procedure

1. Add alcohol to main tank. Start mixing.

2. Pre-dissolve MEA Borate/MIPA Borate in water; add to alcohol.

3. Add isobutylene/ethylmaleimide/hydroxyethylmaleimide copolymer and mix until completely dissolved.

4. Add cyclopentasiloxane; mix until homogeneous.

5. Add the fragrance and ammonium hydroxide separately; mix until uniform.

6. Fill into cans and charge with Hydrofluorocarbon 152a, followed by the A-70.

Packaging

Valve Summit SV-92/97 (0.016″×0.020 Body×0.013″ VT 0.125″ ID)

Actuator V-94, 0.025″ M B

Can Type DOT 2Q Tin-plate.

In combination, the high pressure, high force propellant system and copolymer of the invention provides a 55% VOC hair styling spray composition with the advantages of high hair volumization dimension, definition and texture while permitting easy hair, cutting and styling.

In a preferred embodiment, the composition can include one or more cationic additives such as cationic polymers and cationic surfactants, e.g.,

Ceraphyl 60,

fragrances,

plasticizers and/or shine agents, such as silicones,

phenyl trimethicone, e.g., Si-Tec™ PTM 20

cyclopentasiloxane, e.g., Si-Tec™ CM 040,

corrosion inhibitors, such as

MEA Borate and/or MIPA Borate.

This invention has been described with reference to preferred embodiments in which the polymer is an isobutylene/ethylmaleimide/hydroxyethylmaleimide copolymer, i.e. Aquaflex® FX-64. However, the invention can be practiced with other polymers such as a vinylpyrrolidone/vinyl caprolactam/dimethylaminopropyl methacrylamide copolymer, e.g. Aquaflex® SF-40, or vinylpyrrolidone/vinyl caprolactam/dimethylaminoethyl methacrylate polymer, e.g., GAFFIX® 713, and polyurethanes such as Polyurethane-1. Preferred are polymers having a MW of 20,000-200,000, more preferably 30,000-180,000, and most preferably 40,000-80,000.

While the invention has been described with particular reference to certain embodiments thereof, it will be understood that changes and modifications may be made which are within the skill of the art. Accordingly, it is intended to be bound only by the following claims, in which:

What is claimed is:

1. A hair styling spray composition comprising an isobutylene/ethylmaleimide/hydroxyethylmaleimide copolymer as film-former, water and alcohol, and a propellant admixture of (a) a hydrofluorocarbon and (b) propane/isobutane.

2. A 55% VOC composition according to claim 1 including, by wt., about 4% of said copolymer, about 12% water, about 45% ethanol, and about 31% of (a), and about 6% of (b) in a 50/50 wt. mixture of propane/isobutane.

3. The composition of claim 1 wherein said hydrofluorocarbon is 1,1-difluoroethane.

4. The composition of claim 1 further comprising a cationic polymer or a cationic surfactant.

5. The composition of claim 1 further comprising a corrosion inhibitor.

6. The composition of claim 1 further comprising a silicone selected from phenyl trimethicone and cyclopentasiloxane.
